(19) United States
(12) Patent Application Publication
(10) Pub. No.: US 2016/0002151 A1
(43) Pub. Date: Jan. 7, 2016

(54) METATHESIS PROCESS COMPRISING THE EXTRACTION OF THE ETHYLENE FORMED BY MEANS OF A MEMBRANE

(71) Applicant: ARKEMA FRANCE, Colombes (FR)

(72) Inventors: Jean-Luc DUBOIS, Millery (FR); Jean-Luc COUTURIER, Lyon (FR)

(73) Assignee: ARKEMA FRANCE, Colombes (FR)

(21) Appl. No.: 14/768,023

(22) PCT Filed: Mar. 18, 2014
(86) PCT No.: PCT/FR2014/050628

§ 371 Date: Aug. 14, 2015

(30) Foreign Application Priority Data
Mar. 19, 2013 (FR) 1352413

(51) Int. Cl. C07C 253/30

(52) U.S. Cl. CPC C07C 253/30

(57) ABSTRACT

A process for the metathesis of two α-olefin compounds, wherein it comprises the use of at least one membrane for extracting ethylene from the reaction medium, said membrane being permeable to gases and impermeable to liquids. A process for the metathesis of two α-olefin compounds, carried out in a reaction device including two zones separated by said at least one membrane: a first zone, fed with reactants and catalyst, in which the liquid-phase metathesis reaction is initiated and the liquid reaction medium is circulated in contact with the wall constituted by the membrane, and a second zone, fed with a gaseous stream that is inert with respect to the membrane and the constituents of the reaction medium of the first zone.

The work which led to this invention received financial support from the European Union in the context of Framework Program 7 (FP7/2007-2013) under project No. 241718 EUROBIOREF.

### FIELD OF THE INVENTION

The invention relates to a metathesis process comprising the extraction of the formed ethylene from the reaction medium by means of at least one membrane that is permeable to gases and impermeable liquids.

TECHNICAL BACKGROUND

The cross metathesis reaction is an equilibrated reaction according to the scheme A+B [symbol] C+D, in which the reverse reaction takes place on approaching equilibrium. In general, to obtain the synthesis of the desired final product, C or D in the scheme, the reaction equilibrium is shifted either by supplying one reagent in excess relative to the other, or by removing one of the synthesized products gradually as it is formed.

The route which consists in supplying one reagent in excess has its limits. Specifically, metathesis is a catalytic reaction in which the catalysts which are efficient for the cross metathesis reaction also promote the homometathesis reaction of each of the compounds A and B of the above scheme which always accompanies the main reaction. This leads to mixtures in the reactor that are difficult to separate.

Another problem encountered in metathesis reactions is the deactivation or inhibition of the catalyst under the action of certain metathesis reagents and products, this catalyst being, it should be pointed out, expensive. It would therefore be desirable, in order to avoid rapid deactivation/inhibition of the catalyst, to work with low concentrations of these reagents and/or products.

The metathesis reaction is performed in the liquid phase in the presence of catalysts generally belonging to a family of ruthenium compounds that will be described later. The continuous total separation of compounds C or D in liquid form under the reaction conditions is virtually impossible in the reactor. Specifically, the products are present in liquid form and/or in a form that is soluble in the reaction medium and their boiling point is incompatible with the stability range of the catalyst, which does not make it possible to envisage a continuous distillation. In a certain number of cases, the physicochemical properties of the reagents A or B and of the products C or D are relatively similar. It is therefore preferable to work with a high conversion of the reagents so as not to encounter separation problems. In this context, industrialists choose to perform the metathesis reaction with coreagents A and B which are capable of forming via metathesis gaseous olefinic compounds that can be extracted from the reaction medium by distillation. These olefinic compounds are, for example, ethylene, propylene or butenes, with a preference for ethylene, whose boiling point is the lowest.

When one of the compounds C or D is a light compound, it may partly be removed by “continuous distillation” either by reducing the total pressure (by working under partial vacuum), but this solution also has the risk of accidentally introducing oxygen into the reactor, or by heating the reaction medium to increase its vapor pressure. However, the latter solution reduces the lifetime of the catalyst by thermal deactivation, reduces the selectivity of the cross metathesis relative to the homometathesis reactions and has a tendency to promote isomerization reactions by shifting the double bond. A technical solution that is preferably equally applicable at low temperature and at high temperature is thus sought.

It is known, see in this respect the publication by Jim Patel et al. entitled High conversion and productive catalyst turnovers in cross-metathesis reactions of natural oils with 2-butene published in Green Chem., 2006, August, pp. 450-454, that, in the case where the cross metathesis reaction is performed in the presence of α-olefins as reagent or as product, the catalyst may rapidly lose its activity over time. In the case of metathesis reactions involving ethylene, the catalyst undergoes transformations which greatly inhibit metathesis. This inhibition is believed to be attributable to the formation of a species with the catalyst, for example such as Ru═CH₂, so that the catalyst becomes deactivated very rapidly, thus leading to a substantial loss of catalyst.

To solve this problem, it is necessary to limit the presence in the liquid phase of species containing terminal double bond(s), especially ethylene, dissolved in the reaction medium.

When the reagents themselves contain terminal double bonds, the only possibility is to remove the ethylene formed from the liquid reaction medium. Ethylene is a gas, which is naturally removed from the reaction liquid by distillation due to its low boiling point. However, its solubility in the liquid reaction medium depends on the temperature and is proportionately greater the lower the reaction temperature. It might be envisaged to perform the reaction at a higher temperature in order to reduce its solubility in the medium. However, high temperatures impose other more expensive operating conditions in terms of solvent, operating pressure, degradation of the catalyst, reaction selectivity, etc.

The existing solutions for removing ethylene consist either in entraining the ethylene by distillation at temperatures above 120° C., which has a tendency to bring about thermal degradation of the catalyst, or to work with a reaction medium under partial vacuum in order to remove the ethylene more rapidly, but this solution is not preferred industrially since a vacuum considerably increases the cost of the process. A compromise must thus be found between i) a high temperature that makes it possible to have lower solubilities of ethylene and greater entrainment of gaseous ethylene and ii) a temperature that is low enough to limit the thermal deactivation of the catalyst.

The aim of the present invention is thus to provide a metathesis process in which ethylene is removed as rapidly as possible during the metathesis reaction. In particular, the aim of the present invention is to provide such a process that is capable of functioning at low temperature and at high temperature and that can prevent the inhibition of the metathesis reaction.

The Applicant has now found, entirely surprisingly and unexpectedly, that by circulating the metathesis reaction medium over a membrane system that is permeable to gases makes it possible to withdraw the ethylene formed and partly dissolved in the liquid phase of the reaction medium, to limit the inhibition of the reaction and even to reduce the required amounts of metathesis catalyst while at the same time improving the performance of the metathesis reaction.

It is known practice to use membranes or more generally membrane systems to remove the gases from a liquid or, conversely, to gasify a liquid. Reference may be made in this respect to the article by Miroslav Stanojevic et al. entitled Review of membrane contactors designs and applications of different modules in industry published in FME Transactions vol. 31, No. 2, pp. 91-98 (2003). Alongside the standard applications of degassing or of gasification of aqueous systems, other known industrial applications of these techniques include: the degassing of dielectric or hydraulic organic fluids, the degassing of inkjet printer inks and a whole range of separations via porous or dense membranes in the food industry.

U.S. Pat. No. 6,217,634 describes the use of membrane systems for removing gases formed during the decomposition of dielectric fluids, which may bring about the destruction of transformers. This method is generally used for controlling the behavior of transformers with a view to adapting the industrial treatment for removing light gases by vacuum extraction.

U.S. Pat. No. 8,205,977 describes a similar method for degassing inkjet printer inks to avoid the problems of absence of pixels on printing.

U.S. Pat. No. 6,402,810 describes a process for the membrane-mediated degassing and dehydration of organic hydraulic fluids.

U.S. Pat. No. 6,790,262 describes a membrane device for degassing liquids and more particularly ink.

The abovementioned prior art is solely devoted to the purification of liquids to extract generally gaseous impurities therefrom. The problem to be solved by the process of the invention is not that of removing impurities, but of extracting from the reaction medium, during the reaction, all or part of the ethylene produced so as to improve the performance of the process. The solution to this problem passes via the use of a membrane system implanted in the cross metathesis reaction device.

Most of the membranes used in the applications targeted above in the prior art prove to be unsuitable and do not satisfy the new problem posed by the process of the invention for multiple reasons such as:
i) resistance to diffusion of the gases by single and excessively thick dense membranes,
ii) insufficient permeability to gases, liable to cause “clogging”, slowing down the reaction, or even blocking it,
iii) insufficient chemical stability,
iv) swelling of the membranes at high temperature,
v) chemical incompatibility of the membranes with the solvents, or even with the reagents or the products themselves, reflected by problems of accelerated aging and degradation of the membranes,
vi) inhibition of the catalyst, causing low degrees of conversion for the reaction, etc.

The existing membrane techniques do not satisfactorily solve the abovementioned problems, at the very least because the operating conditions and the various chemical products differ entirely.

The aim of the present invention is thus to provide a novel metathesis process using a system for removing ethylene, which simultaneously has adapted selective permeability, good temperature stability and chemical stability with respect to the various products present in the reaction medium.

### DESCRIPTION OF THE INVENTION

The invention is now described in greater detail and in a nonlimiting manner in the description which follows.

One subject of the present invention is thus a process for the metathesis of two α-olefinic compounds, characterized in that it comprises the extraction of the formed ethylene from the reaction medium by means of at least one membrane that is permeable to gases and impermeable to liquids.

The process of the invention more particularly concerns the metathesis of two α-olefinic compounds and is characterized in that it is performed in a reaction device comprising two zones separated by at least one membrane that is permeable to gases and impermeable to liquids:
a first zone, fed with reagents and catalyst, in which the liquid-phase metathesis reaction is initiated and the liquid reaction medium is placed in circulation in contact with the wall constituted by the membrane; and
a second zone, fed with a stream of gas that is inert toward the membrane and the constituents of the reaction medium of the first zone, optionally under a pressure slightly lower than that prevailing in the first zone or in the form of a flush with a gas stream at a sufficient flow rate, so as to bring about the migration by diffusion of the ethylene dissolved in the reaction medium from the first zone to the second zone.

The process of the invention applies to a metathesis reaction entailing the formation of ethylene. The process of the invention applies especially to the cross metathesis of two functionalized or non-functionalized α-olefinic compounds.

The process of the invention applies more particularly to cross metathesis between an ω-unsaturated functional compound and a functionalized or non-functionalized light α-olefin. Finally, the process of the invention may also be used in the synthesis of a symmetrical difunctional compound by homometathesis of a functional α-olefinic compound.

In the present description, the following definitions apply for the purposes of the invention:
“α-olefinic compound”, a functionalized or non-functionalized molecule, bearing a terminal double bond of formula R₁R₂C═CH₂ in which R₁ and R₂, which may be identical or different, are H, an alkyl, alkenyl or aryl radical comprising from 1 to 14 carbon atoms and optionally comprising a function such as: acid, ester, nitrile, alcohol, aldehyde, etc., it being pointed out that R₁ and R₂ are not simultaneously H;
“light α-olefin” (or light olefin), an olefin comprising from 3 to 5 and preferably from 3 to 4 carbon atoms and optionally bearing one or more acid, ester or nitrile functions such as acrylonitrile, alkyl acrylates, etc.;
“reaction device”, all of the elements, irrespective of their location, in which the liquid reaction medium circulates in a closed circuit.

The process of the invention especially allows the synthesis of monofunctional or difunctional fatty acids/esters. The starting material used in the process of the invention is advantageously an unsaturated fatty acid/ester/nitrile of natural origin in which the double bond(s) are in principle located in position 6, 9, 10, 11, 12, 13 or 15. It is necessary to modify this fatty ester/acid/nitrile by subjecting it especially to a preliminary ethenolysis reaction or thermal cracking (pyrolysis) to give an ω-unsaturated fatty acid/ester/nitrile compound of formula CH₂═CH—(CH₂)ₙ—R in which R is COOH, COOR₃ or CN, R₃ being an alkyl radical comprising from 1 to 11 carbon atoms and n being an integer in the range from 3 to 13.

The cross metathesis reaction is performed in liquid medium in the presence of a catalyst under the following operating conditions. The temperature is generally in the range from 20 to 160° C. and preferably from 20 to 120° C. The pressure is generally in the range from 1 to 30 bar. The reaction is preferably performed at low pressure in the range from 1 to 10 bar and more preferably at atmospheric pressure when the boiling point of the reagents used permits it. Specifically, in so far as an evolution of ethylene is targeted, it is advantageous to work at low pressure, preferably atmospheric pressure. The reaction may be performed without solvent or in the presence of at least one solvent, such as toluene, xylenes or dichloromethane, for example. The reaction is preferably performed without solvent.

The first zone is preferably provided with means for extracting the gaseous phase formed, above the reaction medium, especially the ethylene produced, and also means for extracting said reaction medium at the end of the reaction, on the one hand. The second zone is provided with means for extracting the gas stream enriched in ethylene, on the other hand.

The present invention makes it possible to overcome the drawbacks of the metathesis processes of the prior art by allowing the extraction of the ethylene formed during the metathesis reaction and especially that dissolved in the liquid phase of the reaction medium. The invention more particularly provides a process for the cross metathesis of a functional α-olefinic compound with a light olefin such as propylene or butenes, or with a functionalized olefin such as acrylonitrile or methyl acrylate, leading directly to degrees of conversion that were hitherto impossible to achieve via the conventional metathesis processes.

Preferably, the process of the invention comprises, on the one hand, the continuous removal of the ethylene present in the gaseous phase, and, on the other hand, the simultaneous removal of the ethylene dissolved in the liquid reaction medium by diffusion through the membrane that is permeable only to gases. This double removal makes it possible to increase the reaction yield.

Advantageously, the process of the invention is performed in a reaction device comprising at least two zones separated by a membrane that is permeable to gases and impermeable to liquids.

Industrially, a plurality of membranes or of membrane systems defining, for example, alternatively a reaction zone and an ethylene extraction zone are preferably used for the implementation of the process, since it is important to increase the contact surfaces during the implementation of the process.

There are two main types of membranes, porous or microporous membranes, on the one hand, used for various types of filtration, and dense membranes whose selective permeability is based on a solution-diffusion mechanism. In the process of the invention, at least one dense membrane is used for the separation and selective extraction of ethylene.

Advantageously, the membrane used according to the invention is at least partially formed from polymer. The impermeability to liquids implies the use of at least one “dense” homogeneous polymer which must also allow the diffusion of the ethylene produced by the reaction. The dense polymer membranes may constitute the membrane in its entirety (single-layer membrane), only part of the membrane in the case where the dense polymers are combined with other materials (composite membrane), or alternatively may be supported in the form of a thin film deposited on a porous support membrane which gives the membrane system mechanical strength (asymmetric membrane). In the latter case, the dense membrane is much less thick, which also makes it possible to reduce its gas diffusion resistance. The porous support membranes comprise pores whose size is generally in the range from 0.01 to 50 μm.

Any form of membrane may be used, but it preferably cumulatively satisfies several technical imperatives:
it must be impermeable to the liquids of the reaction medium but have good permeability to the gaseous compounds derived from the reaction;
it must have sufficient mechanical strength to withstand the pressure difference between the liquid zone and the gaseous zone;
it must be inert with respect to the catalysts, reagents and products of the reaction and, correlatively, it must not be sensitive to any physical or chemical action of these agents which might result in a loss of performance,
it must have good temperature resistance to withstand the variations during the reaction process.

The mechanical strength of the membrane is directly linked to its thickness. The thickness of the membranes having a suitable mechanical strength is generally in the range from 5 to 1000 μm, preferably from 5 to 700 μm, preferably from 5 to 300 μm and preferably from 10 to 70 μm. When an asymmetric membrane is used, the total thickness is preferably in the range from 5 to 300 μm and preferably from 10 to 70 μm and the thickness of the dense membrane is generally in the range from 0.01 to 10 μm and preferably from 0.1 to 1 μm.

While respecting the preceding criteria, the choice of the nature of the membrane in the process of the invention depends on the reaction medium, the catalysts, the reagents, the products and the solvent, where appropriate, so as to avoid any interaction with the membrane. The most common consequences, besides transformation of the products formed in the liquid reaction medium, are impairment of the membrane, which might hamper, or even block, the metathesis reaction in the liquid medium.

According to a first embodiment, the process of the invention uses at least one single-layer dense polymer membrane, i.e. a membrane consisting at least partially of a non-porous homogeneous polymer that is permeable to ethylene but impermeable to liquids.

According to a second embodiment, which is an alternative or which may be combined with the preceding embodiment, the process of the invention uses at least one asymmetric polymer membrane comprising a porous layer (preferably made of polymer) that is permeable to gases and liquids, covered with a layer of continuous (non-porous) dense polymer membrane that is permeable to ethylene but impermeable to liquids, preferably of lower thickness than that of the porous layer.

In this second configuration, the gas, due to the reduced thickness of the layer of dense polymer, encounters less resistance to diffusion, which makes it possible to accelerate the diffusion process. This type of mixed asymmetric membrane is thus preferred for the process of the invention.

The form of the membrane used in the process of the present invention is also chosen as a function of the type of reactor used and of the targeted industrial objective. The membrane may be in the form of a flat film, in the form of a tubular or cylindrical film or in the form of a hollow fiber, of suitable dimensions.

The unit hollow fiber (or the tubular film) generally has an outside diameter in the range from 50 to 1000 μm, preferably from 150 to 700 μm and more preferably from 250 to 300 μm. The inside diameter of the fiber is generally in the range from 30 to 300 μm. In the case of the hollow fiber or of the tubular film, the thickness of the membrane is preferably in the range from 10 to 70 μm and that of the dense membrane in the case of an asymmetric membrane is preferably in the range from 0.01 to 10 μm.

The term “module” or “membrane module” refers hereinafter to the assembly formed by one or more membranes. The use of hollow-fiber module(s) and, to a lesser degree, the use of tubular-film or cylindrical-film module(s) constitute a particularly advantageous solution relative to flat membranes since they make it possible to increase the ratio of the exchange surface area to the volume of reaction medium treated, to accelerate the extraction of the ethylene formed and thus to further increase the efficacy of the process of the invention. The process thus preferably uses one or more membranes in the form of hollow fibers, making it possible to have a greater ratio of exchange surface area per unit volume of liquid treated, than in the case of a flat membrane.

According to a first embodiment of the process of the invention, a module in the form of a bundle of hollow fibers is immersed in the reactor in which the metathesis reaction is performed, and the ethylene is withdrawn and extracted by flushing with gas, preferably inside the fiber. Where appropriate, a pressure slightly below that of the reactor is applied on flushing. This naturally supposes that the fibers used have a thermal/mechanical strength that is compatible with the metathesis temperature and pressure operating conditions. It is possible to proceed in the reverse manner, namely to perform the reaction inside each fiber and to perform the extraction of the ethylene by means of the gas stream from the space surrounding the fibers (around the fibers) to which is applied, where appropriate, a pressure difference. However, such a solution is less preferred especially on account of the possible problem of loss of pressure inside the fiber.

According to a second embodiment of the process of the invention, the metathesis reaction is performed in a reactor which is connected (for example at the bottom) to a membrane separation device comprising at least one ethylene extraction module. Part of the liquid flow from the reaction medium is thus sent to the membrane separation device, for example by means of a pump. The liquid flow leaving said device is returned into the reactor for the continuation of the reaction. This type of configuration has the advantage of being able to control the temperature of the liquid reaction medium sent to the membrane separation device. Specifically, in the case where the temperature conditions of the reaction are incompatible with good extraction efficacy, for example an excessively high temperature, two heat exchangers are inserted in the circuit, the first upstream of the separator to lower the temperature of the liquid flow to its operating threshold and the second downstream to raise the temperature of the liquid flow to the temperature level of the reaction medium of the reactor before its recycling.

In the second embodiment, the liquid reaction medium is sent to the membrane separation device. The device is advantageously chosen from several types of modules, and thus the most suitable are especially the flat module, the spiral module or the hollow-fiber module.

According to yet another embodiment of the process of the invention using at least one tubular film (module) (or alternatively a hollow-fiber module), the reaction medium is circulated inside the film (or the fibers) inside the module in which circulates a gas phase that is to be enriched in ethylene. It is also possible to proceed in the reverse manner, i.e. to circulate the extraction gas inside the fibers and the reaction medium in the remaining free space of the module.

Inside the module, the extraction of the ethylene may be performed by co-current, counter-current or cross-current flow, preferably cross-current flow.

Modules of this type make it possible to substantially increase the exchange surface area of the membranes between the feed flow and the extraction flow.

One of the criteria in the choice of polymer(s) for the dense membrane, used either alone as a single layer, or in asymmetric form combined with a porous membrane, is its oleophobic nature. In addition, it is preferably heat-resistant so as to be able to be used at the operating temperatures of the metathesis reaction, without having to apply heat exchanges to the reaction medium. Finally, it is important to check its physical and chemical inertness with respect to the reagents, products or solvents involved in the metathesis reaction.

As examples of polymers that may be included in the composition of the membranes that may be used in the process of the invention, mention may be made of polyolefins such as polyethylene, polypropylene, poly-3-methylbutene-1 and poly(4-methyl-1-pentene) (PMP); vinyl polymers such as polystyrene, PMMA (polymethyl methacrylate); polysulfones; fluorinated or chlorinated polymers such as polyvinylidene fluoride (PVDF), polytetrafluoroethylene (PTFE), fluorovinylethylene/tetrafluoroethylene copolymers, perfluoro-2,2-dimethyl-1,3-dioxole (PDD), perfluoroalkoxy (PFA), polychloroprene (PCP); polyamides such as nylon 6, nylon 66 and nylon 12; polyesters such as polyethylene terephthalate, polybutene terephthalate and polyethylene-2,6-naphthalate; polycarbonates such as poly-4,4′-dihydroxydiphenyl-2,2-propane carbonate; polyethers such as polyoxymethylene and polymethylene sulfide; polyphenylene chalcogenides such as polythioether, polyphenylene oxide and polyphenylene sulfide; polyether ether ketones (PEEK), polyether ketone ketone (PEKK), silicones such as polyvinyltrimethylsiloxane (PVTMS), polydimethylsiloxane (PDMS), perfluoroalkoxy (PFA) and polychloroprene (PCP).

Among the polymers mentioned above, perfluoro-2,2-dimethyl-1,3-dioxole (PDD), polytetrafluoroethylene (PTFE) and perfluoroalkoxy (PFA), and mixtures thereof, are preferred for the composition of the membrane according to the process of the invention.

Hollow fibers that are particularly suitable for the invention may be manufactured using, in combination with the dense membrane, various porous materials, such as polypropylene, polyvinylidene fluoride (PVDF) or polysulfone. By way of example, the interior and/or the exterior of the hollow fibers is coated with a dense thin layer whose thickness may range from 0.01 to 10 μm of an amorphous copolymer of perfluoro-2,2-dimethyl-1,3-dioxole (PDD) and variable amounts of tetrafluoroethylene (TFE). Other fluoro polymers may also be used successfully. For example, Teflon® AF 2400 and Teflon® AF 1600 (EI from Pont de Nemours and Co) are two copolymers that are suitable for use in the process of the invention. This layer forms a barrier to the organic reaction medium and has good mechanical strength, while at the same time being sufficiently permeable to ethylene to allow its rapid diffusion toward the gaseous zone.

The metathesis reaction is performed in the presence of at least one metathesis catalyst. These catalysts are well known and an entire range of them exists. Mention may, for example, be made of the tungsten complexes developed by Schrock et al (J. Am. Chem. Soc. 108:2771, 1986) or Basset et al. (Angew. Chem., Ed. Engl. 31:628, 1992). More recently, catalysts termed Grubbs catalysts have emerged (see Grubbs et al., Angew. Chem., Ed. Engl. 34:2039, 1995 and Organic Letters 1:953, 1999) which are ruthenium-benzylidene complexes operating in homogeneous catalysis. Other studies have been carried out in order to produce immobilized catalysts, i.e. catalysts of which the active principle is that of the homogeneous catalyst, in particular ruthenium-carbene complexes immobilized on an inactive support.

The process according to the invention advantageously uses at least one metathesis catalyst of ruthenium-carbene type. Said ruthenium-carbene catalyst is preferably chosen from charged or uncharged catalysts of general formula:

(X₁)ₐ (X₂)_{b}Ru(carbene C) (L₁)_{c}(L₂)_{d} (L₃)ₑ

in which:
a, b, c, d and e are integers, which may be identical or different, with a and b equal to 0, 1 or 2; c, d and e equal to 0, 1, 2, 3 or 4;
X₁ and X₂, which may be identical or different, each represent a charged or uncharged and monochelating or polychelating ligand; by way of examples, mention may be made of halides, sulfate, carbonate, carboxylates, alkoxides, phenoxides, amides, tosylate, hexafluorophosphate, tetrafluoroborate, bis(triflyl)amide, an alkyl, tetraphenylborate and derivatives; X₁ or X₂ can be bonded to L₁ or L₂ or to the carbene C so as to form a bidentate or chelate ligand on the ruthenium; and
L₁, L₂ and L₃, which may be identical or different, are electron-donating ligands, such as phosphine, phosphite, phosphonite, phosphinite, arsine, stilbene, an olefin or an aromatic compound, a carbonyl compound, an ether, an alcohol, an amine, a pyridine or derivative, an imine, a thioether, or a heterocyclic carbene; L₁, L₂ or L₃ can be bonded to the carbene C so as to form a bidentate or chelate ligand, or a tridentate ligand.

The carbene C is represented by the general formula: CR₁R₂ for which R₁ and R₂ are groups which may be identical or different, such as hydrogen or any other functionalized or non-functionalized hydrocarbon-based group of saturated, unsaturated, cyclic, aromatic, branched and/or linear type. By way of examples, mention may be made of ruthenium alkylidene, benzylidene, benzylidene ether or cumylene complexes, such as vinylidenes Ru═C═CHR or allenylidenes Ru═C═C═CR₁R₂ or indenylidenes.

A functional group (making it possible to improve the retention of the ruthenium complex in an ionic liquid) can be grafted onto at least one of the ligands X₁, X₂, L₁, L₂, or onto the carbene C. This functional group may be charged or uncharged, such as preferably an ester, an ether, a thiol, an acid, an alcohol, an amine, a nitrogenous heterocycle, a sulfonate, a carboxylate, a quaternary ammonium, a guanidinium, a quaternary phosphonium, a pyridinium, an imidazolium, a morpholinium or a sulfonium.

The metathesis catalyst can optionally be rendered heterogeneous on a support in order to facilitate the recovery/recycling thereof.

The cross metathesis catalysts of the process of the invention are preferably ruthenium carbenes described, for example, in Aldrichimica Acta, vol. 40, no. 2, 2007, p. 45-52.

Examples of such catalysts are Grubbs catalysts, Hoveyda-Grubbs catalysts, Piers-Grubbs catalysts, and other metathesis catalysts of the same type, whether they are “1st-generation”, “2nd-generation” or “3rd-generation” catalysts.

Grubbs catalysts are based on a ruthenium atom surrounded by 5 ligands:
2 anionic ligands, such as halides;
2 electron-donating ligands, such as trialkyl phosphines, or saturated N-heterocyclic carbenes (called NHC ligands);
an alkylidene group, such as substituted or unsubstituted methylene groups ═CR₂.

These metathesis catalysts are classified into two categories, depending on the nature of their electron-donating ligands L:
those which contain two phosphine ligands (and no saturated NHC ligand), developed first, are 1st-generation-type catalysts;
those which contain a saturated NHC ligand (heterocyclic carbene) are 2nd-generation-type catalysts.

A type of catalyst known as a “Hoveyda-Grubbs” catalyst contains, among the electron-donating ligands, a benzylidene-ether chelating ligand, and either a phosphine (1st generation) or a saturated NHC ligand (2nd generation), usually substituted with phenyls generally substituted with mesityl (Mes) groups or else with isopropyl (iPr) groups.

Another type of catalyst termed “Piers-Grubbs” catalyst forms a four-ligand cationic complex which does not require dissociation of a ligand before the reaction.

Other types of catalysts are the “Umicore”, “Zanan” and “Grela” catalysts.

Generally, the choice of the catalyst depends on the reaction under consideration.

According to an advantageous embodiment, the catalyst is free of phosphine.

Preferred catalysts are the catalysts which follow:

(1) The catalyst denoted “Hoveyda-Grubbs 2”, having the following formula:

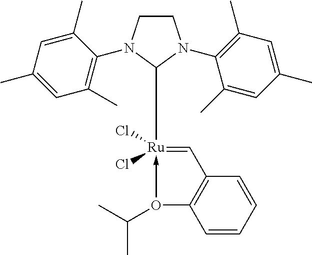

(2) The catalyst denoted “M51”. having the following formula:

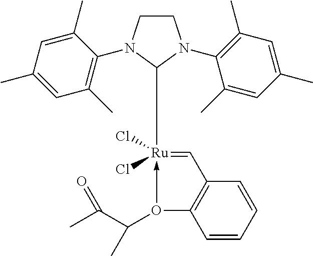

(3) The catalyst denoted “M71-SIPr”, having the following formula:

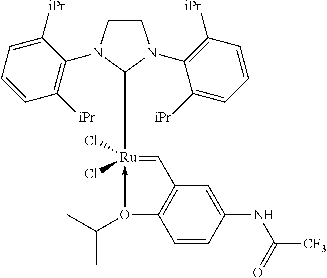

(4) The catalyst denoted “M71-SIMes”, having the following formula:

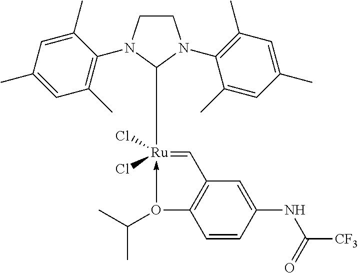

(5) The catalyst denoted “M72-SIPr”, having the following formula:

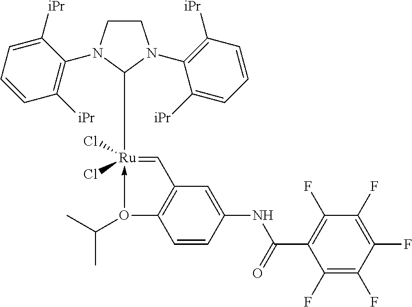

(6) The catalyst denoted “M73-SIPr”, having the following formula:

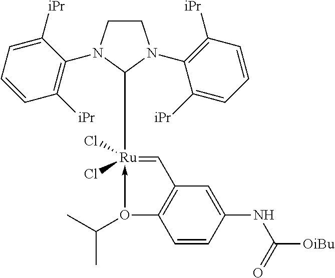

(7) The catalyst denoted “M74-SIPr”, having the following formula:

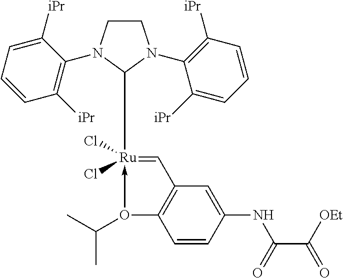

(8) The catalyst denoted “Nitro-Grela-SIMes”, having the following formula:

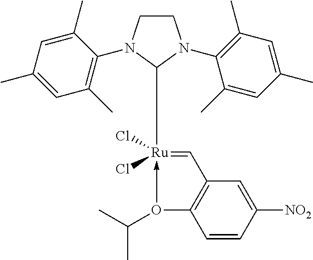

(9) The catalyst denoted “Nitro-Grela-SIPr”, having the following formula:

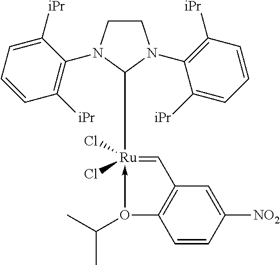

(10) The catalyst denoted “Apeiron AS2034”, having the following formula:

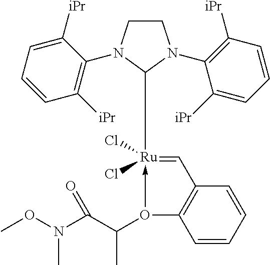

(11) The catalyst denoted “Zannan 44-0082 (Strem)”, having the following formula:

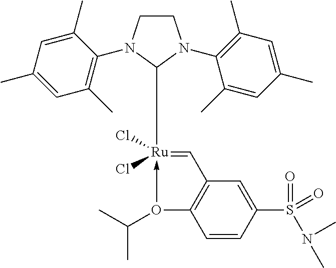

(12) The catalyst denoted “M831-SIPr”, having the following formula:

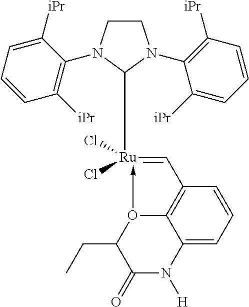

(13) The catalyst denoted “M832-SIPr”, having the following formula:

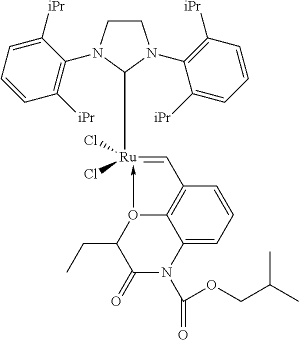

(14) The catalyst denoted “M853-SIPr”, having the following formula:

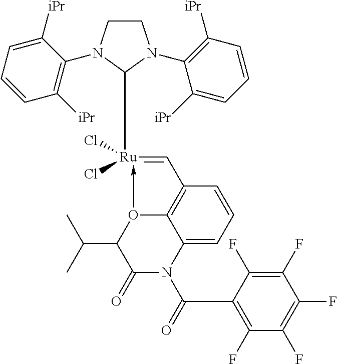

(15) The catalyst denoted “M863-SIPr”, having the following formula:

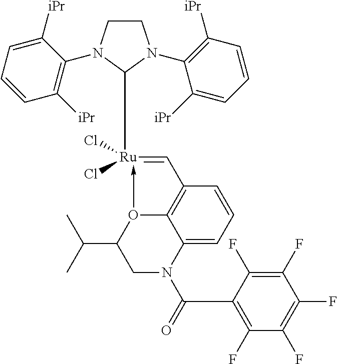

(16) The catalyst denoted “Materia C711”, having the following formula:

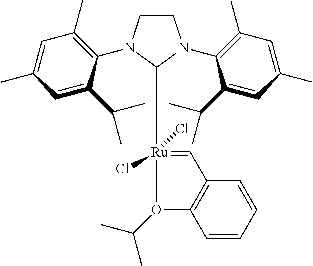

### EXAMPLES

The following examples illustrate the invention without limiting it.

In the examples that follow, purified methyl 10-undecenoate and an olefin, acrylonitrile, are used as reagents and M71SiPr (sold by Umicore) is used as catalyst: 10.2 mg are dissolved in 21.6 g of toluene. 5 ml of this solution are added, i.e. 2.02 mg of catalyst.

In the process of Example 1 according to the invention, a PF13F module from DIC is used. This is a dense PTFE membrane.

In the process of Comparative Examples 2 and 3, the procedure is performed without a membrane.

The process is performed as follows, and the following results are obtained.

### Description of the Equipment

The equipment is composed of a 600 mL round-bottomed flask equipped with a nitrogen inlet, a temperature probe, a condenser for condensing and recycling in the reactor especially solvent and acrylonitrile (on which condenser is mounted a tap and which exits to a bubbler), an oil bath and an inlet for reagents and the catalyst added continuously.

The reaction medium is withdrawn continuously by a pump, placed downstream of a heat exchanger for bringing the temperature of the reaction medium to 40° C. and which pushes the liquid through the membrane module. The liquid leaving the module also passes through an exchanger which brings the liquid to the reaction temperature, and the liquid is recirculated into the reactor. The module is maintained under a partial vacuum of 60 kPa absolute. The circulation rate of the liquid is 200 ml/minute.

Procedure followed:
Purge the equipment with nitrogen. Next, switch off the nitrogen during the loading of the acrylonitrile.
Load into the reactor 150 g of toluene.
Load into the reactor:
15 g of purified (by adsorption on alumina) methyl 10-undecenoate
2.0 g of acrylonitrile.
Heat to 110° C. while leaving the condenser tap open, and monitor the temperature of the medium. (oil bath at 145° C.)
Weigh out 10.2 mg of catalyst (M71SiPr from Umicore) in a glass crucible. Dilute it in 21.6 g of toluene in a Schlenk tube under nitrogen. 5 ml of the solution, i.e. 2.02 mg of catalyst, are introduced into the syringe, which is placed on the syringe pump, and the catalyst is prepared before loading the acrylonitrile.
Leave the flow of nitrogen on during the preparation of the catalyst and switch it off during loading.—Fill a syringe with about 2.4 g of acrylonitrile and place it on the second syringe pump.
Take a sample at T=0.
Initiate the start of the reaction at T=0 with the continuous addition of catalyst and acrylonitrile over 2 hours.
Take a sample at T=10 min, 20 min, 30 min, etc. and 120 min and perform

GC analysis on the sample.
Stop the heating, the condenser bath and leave to cool.

### Example 1

After 2 hours, the yield of nitrile ester obtained is 75 mol %.

### Comparative Example 2

The same reaction is performed in the absence of membrane module, all the conditions being otherwise equal.

After the 2 hours of reaction, the yield is 68 mol %.

### Comparative Example 3

The test of Example 2 is repeated using double the amount of catalyst (M71SiPr). After the 2 hours of reaction, the yield is 74 mol %.

1. A process for metathesis of two α-olefinic compounds in a metathesis reactor, the process comprising the extraction of the formed ethylene from a reaction medium by means of at least one membrane that is permeable to gases and impermeable to liquids.

2. The process as claimed in claim 1, wherein the process is performed in a reaction device comprising two zones separated by said at least one membrane:
a first zone, fed with reagents and catalyst, in which the liquid-phase metathesis reaction is initiated and the liquid reaction medium is placed in circulation in contact with the wall constituted by the at least one membrane, and
a second zone, fed with a stream of gas that is inert toward the at least one membrane and the constituents of the reaction medium of the first zone, optionally under a pressure slightly lower than that prevailing in the first zone or in the form of a flush with a gas stream at a sufficient flow rate, so as to bring about migration by diffusion of the ethylene dissolved in the reaction medium from the first zone to the second zone.

3. The process as claimed in claim 1 wherein said at least one membrane has a thickness in the range from 5 to 1000 μm.

4. The process as claimed in claim 1, wherein said at least one membrane comprises at least one layer comprising at least one dense polymer.

5. The process as claimed in claim 1, wherein said at least one membrane is asymmetrical and comprises a porous layer and a dense polymer layer, in which the total thickness of said asymmetrical membrane is in the range from 5 to 300 μm, the thickness of the dense membrane being in the range from 0.01 to 10 μm.

6. The process as claimed in claim 1, wherein said at least one membrane is in the form of a hollow fiber.

7. The process as claimed in claim 6, in which the hollow fiber has an outside diameter in the range from 50 to 1000 μm, and an inside diameter of the fiber in the range from 30 to 300 μm.

8. The process as claimed in claim 6, wherein the process is performed in the presence of a bundle of hollow fibers immersed in the metathesis reactor and by means of which ethylene is withdrawn and extracted by flushing with gas.

9. The process as claimed in claim 1, wherein the metathesis reactor is connected to a membrane separation device comprising at least one ethylene extraction module by means of which at least one liquid flow derived from the reaction medium is sent to the membrane separation device for extracting ethylene and at least one liquid flow exiting said membrane separation device is returned into the metathesis reactor for the continuation of the reaction.

10. The process as claimed in claim 9, the process further comprising using two heat exchangers, a first heat exchanger being upstream of the membrane separation device for lowering the temperature of the liquid flow to an operating threshold of the membrane separator, and a second heat exchanger being downstream of the membrane separation device to raise the temperature of the exiting liquid flow to the temperature level of the reaction medium of the metathesis reactor.

11. The process as claimed in claim 1, wherein the polymers used in the composition of said at least one membrane are chosen from oleophobic polymers poly-3-methyl; vinyl polymers; fluorinated or chlorinated polymers; polyamides; polyesters; polycarbonates; polyethers; polyphenylene chalcogenides; polyether ether ketones (PEEK), polyether ketone cetone (PEKK), and silicones.

12. The process as claimed in claim 11, wherein the dense membrane comprises at least one polymer chosen from polytetrafluoroethylene (PTFE) and perfluoroalkoxy (PFA), and mixtures thereof.

13. The process as claimed in claim 1, wherein the metathesis reaction is performed in liquid medium in the presence of a catalyst at a temperature in the range from 20 to 160° C. and at a pressure in the range from 1 to 30 bar.

14. The process as claimed in claim 1, wherein the reaction is performed in the presence of at least one solvent chosen from toluene, xylenes and dichloromethane.

15. The process as claimed in claim 1, wherein the reaction is performed in the presence of a metathesis catalyst of ruthenium-carbene type, said ruthenium-carbene catalyst being chosen from the charged or uncharged catalysts of general formula:
(X₁)ₐ (X₂)_{b}Ru(carbene C) (L₁)_{c}(L₂)_{d} (L₃)ₑ
in which:
a, b, c, d and e are integers, which may be identical or different, with a and b equal to 0, 1 or 2; c, d and e equal to 0, 1, 2, 3 or 4;
X₁ and X₂, which may be identical or different, each represent a charged or uncharged and mono-chelating or polychelating ligand;
X₁ or X₂ may be linked to L₁ or L₂ or to the carbene C so as to form a bidentate ligand or chelate on the ruthenium;
L₁, L₂ and L₃, which may be identical or different, are electron-donating ligands; L₁, L₂ or L₃ can be bonded to the carbene C so as to form a bidentate or chelate ligand, or a tridentate ligand
carbene C is represented by the general formula: CR₁R₂ for which R₁ and R₂ are groups which may be identical or different.

16. The process as claimed in claim 15, wherein the catalyst is chosen from alkylidene, benzylidene, benzylidene ether or cumylene ruthenium complexes.
